(19) United States
(12) Patent Application Publication
(10) Pub. No.: US 2004/0146503 A1
(43) Pub. Date: Jul. 29, 2004

(54) Methods and compositions for inducing stable immune tolerance

(76) Inventors: Raymond J. Tesi, Menlo Park, CA (US); Roland Buelow, Mountain View, CA (US)

Correspondence Address: Todd A. Lorenz, DORSEY & WHITNEY LLP, Suite 3400, Four Embarcadero Center, San Francisco, CA, 94111-4187, US

(21) Appl. No.: 10/653,012

(22) Filed: Aug. 28, 2003

Related U.S. Application Data
(60) Provisional application No. 60/406,705, filed on Aug. 28, 2002.

(51) Int. Cl. A61K 39/395; A61K 31/4745; A61K 31/225

(52) U.S. Cl. 424/131.1; 514/291; 514/548

(57) ABSTRACT

The invention relates to immunotherapy, and more specifically, to the use of a novel combination of immunological agents to induce stable immune tolerance to donor or host antigens in recipients of donor organs, tissues and cells.

### FIELD OF THE INVENTION

The field of this invention relates to immunotherapy, and more specifically, to the use of a novel combination of immunological agents to induce stable immune tolerance to donor or host antigens in recipients of donor organs, tissues and cells.

### BACKGROUND OF THE INVENTION

The transplantation of allogeneic organs, tissues and cells has become increasingly important for the treatment of a wide variety of degenerative diseases and malignancies. The use of such allografts, however, is limited by graft rejection caused by antigenic differences between the donor and recipient, primarily involving antigens of the Major Histocompatibility Complex (“MHC”). Without treatment, an allograft generally survives for a period of days to weeks. However, it will eventually become inflamed and infiltrated with lymphocytes and monocytes, leading to necrosis and loss of the graft.

Successful transplantation of non-lymphoid donor tissues and solid organs (solid organ transplantation, or “SOT”) therefore depends on preventing the host immune response to donor antigen. Conversely, the donor immune response against host cells and tissues, known as graft versus host disease or GVHD, must be avoided in transplants of donor lymphoid cells and tissue, such as bone marrow transplantation (“BMT”). GVHD is caused by circulating donor T cells within the host which are acquired in bone marrow grafts. Gale and Butturini, Bone Marrow Transplant 3:185 (1988).

At present, the standard and accepted therapy for prolonging graft survival or treating GVHD involves the chronic administration of immunosuppressive agents such as cyclosporin A (CsA), tacrolimus (FK-506) and sirolimus (Rapamycin) along with azathioprine (Imuran®), mycophenylate mofetil (CellCept®), anti-thymocyte globulins (“ATGs”), monoclonal antibody preparations such as OKT3 and/or corticosteroids such as prednisone. These agents are typically utilized in various combinations for induction and maintenance of immunosuppression, with CsA and FK-506 being the primary long-term immunosuppressive agents. By permanently suppressing the recipient's general immune response, survival of the graft can be enhanced.

Chronic immunosuppressive therapy comes with a steep price, however, both economically and physiologically. The yearly cost for maintenance immunosuppressive therapy can run as much as $10,000. More importantly, the long-term use of cyclosporin and tacrolimus is associated with significant nephrotoxicity, and constant non-specific immune suppression in the host also greatly increases the risk of adventitious infection and related malignancies, such as post-transplant lymphoproliferative disorder.

Moreover, although current therapies have proven successful in dealing with episodes of acute rejection, chronic graft failure is still a major limitation and is the principal cause of graft loss and late death. See Libby and Pober, Immunity 14:387-97 (2001). For example, despite one-year graft survival rates of almost 90%, up to 50% of renal allografts discontinue to function after approximately twelve years. Hariharan et al., N. Engl. J Med. 342:606-12 (2000). Accordingly, the establishment of solid organ transplantation as a successful long-term therapy requires significant improvement in addressing chronic graft deterioration.

A major goal in transplant immunobiology is the induction of stable and specific immunological tolerance to transplanted tissue and organs, with the potential of freeing patients from the side effects of continuous pharmacologic immunosuppression and its attendant complications and costs. Achieving stable and specific immunological tolerance can improve transplant longevity and quality of life for the recipient, and at the same time considerably improve the cost effectiveness of transplant therapy. To date, however, proposed tolerance protocols demonstrating efficacy in small animal models have had limited success when transferred to primate models and human clinical trials. The poor success of these prior art protocols in combination with the severe shortage of donor organs has prevented transplant clinicians from accepting tolerance as a viable alternative to chronic immunosuppressive therapy.

What is needed, therefore, is an immunological tolerance protocol demonstrating efficacy and long-term stability in primates as well as in small animal models. Such a protocol will also have to overcome the significant clinical reluctance to risk extremely scarce resources on new therapies, and further ensure against graft loss due to acute rejection, such that the transplant clinician will have a measure of confidence in utilizing the tolerance protocol in lieu of chronic immunosuppressive therapies.

Relevant Literature

Chiffoleau et al., J Immunol. 168:5058 (2002) describes the induction of tolerance in a fully MHC-mismatched heart allograft model by treatment with LF15-0195. Hubbard et al. Hum. Immunol. 62:479 (2001) describes inducement of long-term tolerance in rhesus monkeys using anti-CD3ε immunotoxin in combination with deoxyspergualin. Contreras et al., Transplantation 65:1159(1998) describes a peritransplant induction strategy involving anti-CD3 immunotoxin supplemented with 15-deoxyspergualin and methylprednisolone wherein treatment induces long-term kidney allograft acceptance. Banchereau et al., Nature 392:245 (1998) illustrates the role of dendritic cells in tolerizing T cells to antigens innate to the body.

Thomas et al., Immunol. Rev. 183:223 (2001); Thomas et al., Diabetes 50:1227 (2001); and Thomas et al., Transplant 68:1660-1673 (1999), describe the authors' efforts to achieve immunologic tolerance via the administration of 15-deoxyspergualin (DSG) and anti-CD3 immunotoxin in various transplant models. In Transplantation 68:11 (1999), Thomas et al. suggest that 15-deoxyspergualin arrests dendritic cell maturation. Thomas et al., Ann NY Acad Sci 685:175(1993) describes the potential use of 15-deoxyspergualin as an immunosuppressive drug in allogeneic transplantation.

U.S. Pat. No. 6,103,235 describes a proposed immune tolerance protocol utilizing immunotoxin (IT) therapy either alone or in combination with other immunosuppressive agents. U.S. Pat. No. 5,762,927 describes a tolerance approach utilizing immunotoxin therapy combined with direct thymic injection of donor lymphocytes.

Neuhas et al., Liver Transpl. 7:473-84 (2001) review proposed mechanisms of action as well as recent preclinical and clinical results of mTOR inhibitors. Wells et al., Nat. Med. 5:1303-7 (1999) suggest that deletion of activated T cells through activation-induced cell death or growth factor withdrawal is necessary to achieve peripheral tolerance across major histocompatibility complex barriers, while Li et al., Nat. Med. 5:1298-1302 (1999) report that the induction of T-cell apoptosis and peripheral allograft tolerance is prevented by blocking both signal 1 and signal 2 of T cell activation.

Hayamizu et al., Transplant Proc. 31:25S (1999) shows that donor cells facilitate tolerance to rat heart allografts subsequent to posttransplant total lymphoid irradiation and the administration of rabbit anti-thymocyte globulin. Ghanekar et al., Transplant Proc. 33:3849 (2001) describes that administration of rabbit antithymocyte serum permits a cyclophosphamide-free approach to prevention of hDAF transgenic porcine kidney xenograft rejection in primates. Knechtle et al., Transplant Proc. 31:27S (1999) describes immunological tolerance induced by administration of anti-CD3 immunotoxin, CTLA4-Ig, and anti-CD40 ligand. Kirk, A. D. Crit Rev Immunol. 19:349 (1999) summarizes techniques for tolerance induction investigated in nonhuman primates.

### SUMMARY OF THE INVENTION

It is an object of this invention to provide methods for inducing stable immunological tolerance in transplant recipients. To this end, the invention provides a novel tolerance protocol for preventing and inhibiting host rejection responses by inducing stable immunological tolerance in transplant recipients to donor or host antigens, comprising the administration to the transplant recipient of a lymphocyte depleter and a deoxyspergualin (“DSG”) compound, in further combination with an mTOR inhibitor to serve as a bridge between transplant and tolerance. Also contemplated by the present invention are methods for treating GVHD and autoimmune diseases.

In a preferred embodiment, the lymphocyte depleter comprises an anti-thymocyte globulin. In a particularly preferred embodiment, the anti-thymocyte globulin comprises a rabbit polyclonal anti-thymocyte globulin, such as Thymoglobulin® available from SangStat Medical Corporation, Fremont, Calif. In an alternative embodiment, the lymphocyte depleter comprises a monoclonal antibody preparation directed against a T cell surface protein such as, e.g., an anti-CD52 monoclonal antibody (e.g., Campath®,). In a still further embodiment, the lymphocyte depleter comprises a T cell immunotoxin such as an anti-CD3 immunotoxin or F(Ab)₂ immunotoxin. See, e.g., Thomas et al., Transplantation 68:1660-73 (1999).

In one embodiment, the deoxyspergualin compound comprises 15-deoxyspergualin (Gusperimus®, Bristol Myers Squibb, Seattle, Wash.). In a preferred embodiment, the deoxyspergualin compound comprises DSG analogue LF15-0195 (Anisperimus™, Laboratoires Fournier, DAIX, FR). In an alternative embodiment, the deoxyspergualin compound comprises DSG analogue LF-08-0299 (Tresperimus®, Laboratoires Fournier, DAIX, FR). In another embodiment the deoxyspergualin compound comprises DSG analogue methyldeoxyspergualin. See, e.g., Odaka et al., Immunology 95:370-6 (1998).

In a preferred embodiment, the mTOR inhibitor comprises sirolimus (Rapamycin, Wyeth-Ayerst, N.J.). In an alternative embodiment, the mTOR inhibitor comprises everolimus (Novartis AG, Basel, CH).

Other objects and advantages of the present invention will be apparent from the description which follows.

### DETAILED DESCRIPTION OF THE INVENTION

In accordance with the subject invention, methods are provided for inducing stable and specific immunological tolerance to either donor or host antigens, depending on the nature of the allograft, in order to prolong the survival of allogeneic grafts without the need for chronic immunosuppressive therapy. As used herein, the phrase “stable immunological tolerance” means stable, long-term graft survival and/or function for at least one year without the use of general immunosuppressive therapy. As used herein, the phrase “specific immunological tolerance” means the absence of a host cellular and/or humoral immune response to donor antigen, combined with evidence of in vivo immune competence to other foreign antigens. The subject protocol may also be utilized to treat GVHD, as well as autoimmune diseases such as diabetes, systemic lupus erythematosus, rheumatoid arthritis, and the like.

The objects of the present invention are achieved by subjecting a recipient host to a novel tolerance protocol comprising the simultaneous or sequential administration of a lymphocyte depleter and a deoxyspergualin compound, in combination with the preferably tapered administration of an mTOR inhibitor to serve as a bridge between transplant and tolerance. By “tapered administration” is meant the gradual reduction and eventual elimination of the mTOR inhibitor, either over a fixed period of time or as determined empirically by the transplant clinician based on regular monitoring of biological markers for graft function and/or survival.

The lymphocyte depleter in the subject protocol serves to substantially deplete T cells from peripheral blood and/or lymphoid tissues. In a preferred embodiment, the lymphocyte depleter comprises an anti-thymocyte globulin (“ATG”) or an anti-lymphocyte globulin. In a particularly preferred embodiment, the anti-thymocyte globulin comprises a rabbit polyclonal anti-thymocyte globulin, such as Thymoglobulin® (SangStat Medical Corporation, Fremont, Calif.). Contrary to recent reports, which have generally disparaged the use of such ATGs for this purpose, see Contreras et al., supra, the present inventors find that polyclonal preparations such as Thymoglobulin® can an effective and preferred lymphocyte depleter for use in the subject protocol.

In another embodiment, the lymphocyte depleter may comprise a monoclonal antibody capable of binding to a receptor or other peptide expressed on lymphocytes, and more preferably on resting and/or activated T cells, such as anti-CD52 monoclonal antibodies (e.g., Campath®)

In an alternative embodiment, the lymphocyte depleter may comprise a T cell immunotoxin such as an anti-CD3 immunotoxin or F(Ab)₂ immunotoxin (“Anti-CD3-IT”). See, e.g., Thomas et al., Transplantation 68:1660-73 (1999). In one such embodiment, the anti-CD-3 IT comprises a α-CD3-IT, a conjugate of murine IgG, anti-rhesus CD3ε (FN18) and the mutated diptheria toxin CRM9, prepared as described in Neville et al., J. Immunothera. Emphasis Tumor Immunol. 19:85 (1996); see also U.S. Pat. Nos. 5,725,857 and 5,167,956, the disclosures of which are expressly incorporated by reference herein in their entirety. Significantly, however, the use of such compounds is less preferred to ATGs in the subject protocol in view of potential complications associated with the use of immunotoxins, including vascular leak syndrome. See Chatenoud, Transplant Proc. 26:3191 (1994).

The subject protocol further comprises the contemporaneous administration of a deoxyspergualin compound. By the phrase “deoxyspergualin compound” or “DSG compound” is meant 15-deoxyspergualin (15-DSG) and active analogues thereof. 15-DSG is a derivative of the antibiotic spergualin, a fermentation product isolated from Bacillus laterosporus. Originally investigated as a potential chemotherapeutic agent, 15-DSG was subsequently shown by a number of investigators to have potent immunosuppressive properties. Umezawa et al., J Antibiot. 38:283-84 (1985); Thomas et al., Annals. N.Y. Acad. Sci. 685:175-192 (1993).

Also contemplated within the scope of deoxyspergualin compounds for use in the subject protocol are bioactive analogues of 15-DSG, including DSG analogues LF15-0195 (Anisperimus™) and LF-08-0299 (Tresperimus®) available from Laboratoires Fournier, DAIX, France. See, e.g., U.S. Pat. No. 5,476,613, No. 5,637,613, No. 5, 733, 928 and No. 5,883,870, the disclosures of which are incorporated by reference herein in their entirety. Also included is methyldeoxyspergualin, recently described in Odaka et al., Immunology 95:370-6 (1998).

The relevant biological activity of 15-DSG is known to result at least in part from its indirect blockade of NF-KB activity via the binding of DSG to the NF-KB chaperon HSC 70, which prevents nuclear translocation of NF-KB to the nucleus where it would normally activate genes involved in dendritic cell maturation and pro-inflammatory cytokine production. Suitable bioactive analogues of 15-DSG for use in the subject protocol would have similar or improved activity in relation to inhibition of dendritic cell maturation and pro-inflammatory cytokine release.

Without being bound by theory, the DSG component of the subject protocol serves to inhibit the maturation of dendritic cells and block the release of proinflammatory Th1-type cytokines such as, e.g., IL-2, IL-12, interferon gamma (IFN-γ), tumor necrosis factor alpha (TNF-α), and the like. Moreover, DSG administration may also increase systemic IL-10, IL-4 and TGF-β production, thereby contributing to the development of a Th2-like cytokine milieu that favors development of specific unresponsiveness in the newly-emerging T cells after the depletion cycle of the subject protocol. See, e.g., Thomas et al., Transplantation 68:1660-73 (1999). DSG administration also leads to a profound inhibition of the host antibody response. Thomas, et al., Annals. N.Y. Acad. Sci. 685:175-192 (1993). This latter effect of DSG therapy is particularly synergistic when used in the preferred embodiment of the subject protocol combining DSG administration with ATG as the lymphocyte depleter, since it also serves to prevent the host humoral response against the xenogeneic immunoglobulin present in the ATG preparations.

The third component of the subject tolerance protocol, the immunosuppressant taper, provides a critical bridge between transplant and tolerance, providing established pharmacological activity against acute rejection to ensure the viability of the graft. As a result of historical difficulties in successfully transferring proposed tolerance strategies from inbred murine models to larger outbred animals in protocols suited for humans, there is significant reluctance in the clinical transplant community to risk extremely scarce donor tissue or organs on anything other than established immunosuppressive regimens. The third component of the subject therapy thus serves as a critical safety net to overcome clinical reluctance and ensure graft survival.

Significantly, however, the selection of an appropriate agent for use as an immunosuppressant taper is complicated by the complex biological mechanisms involved in inducing stable tolerance. Recent evidence indicates that calcineurin inhibitors such as CsA and FK-506, which inhibit the signal I pathway of T cell activation (G₀ to G₁ transition, transcription and cytokine and growth factor gene activation), may actually be counterproductive in tolerance protocols. Li et al., Nat. Med. 5:1298-1302 (1999). Thus, the present invention contemplates instead the use of mTOR inhibitors such as rapamycin and its related analogues, which act to inhibit the signal III pathway (G₁ to S transition, translation and cytokine-driven T cell proliferation). Id.; see Neuhas et al., Liver Transpl. 7:473-484 (2001).

As used herein the term “mTOR inhibitor” includes the neutral tricyclic compound rapamycin and other rapamycin compounds, including, e.g., rapamycin derivatives, rapamycin analogues and other macrolide compounds which are thought to have the same mechanism of action (e.g., inhibition of mTOR activity) These include compounds with a structural similarity to rapamycin, e.g., compounds with a similar macrocyclic structure which have been modified to enhance therapeutic benefit. In a particularly preferred embodiment, the mTOR inhibitor comprises rapamycin (sirolimus, Wyeth-Ayerst, Princeton, N.J.). In another preferred embodiment, the mTOR inhibitor comprises the rapamycin derivative 40-O-(2-hydroxy)ethyl-rapamycin (everolimus, Novartis AG, Basel, CH). In a further embodiment, the mTOR inhibitor comprises the rapamycin ester cell cycle inhibitor-779 (CCI-779).

Additional rapamycin compounds which may be used in the invention are well known in the art and include but are not limited to the rapamycin analogues and derivatives described in U.S. Pat. Nos. 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285;5,504,291; 5,504,204; 5,491,231; 5,489,680; 5,489,595; 5,488;054; 5,486,524; 5,486,523; 5,486,522; 5,484,791; 5,484,790; 5,480,989; 5,480,988,5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203;5,169,851; 5,164,399; 5,162,333; 5,151,413; 5,138,051; 5,130,307;5,120,842; 5,120,727; 5,120,726; 5,120,725; 5,118;678; 5,118,677; 5,100,883; 5,023,264; 5,023,263; 5,023,262; all of which are incorporated herein by reference.

In a further embodiment, donor antigen in the form of donor stem cells or other donor hematopoietic cells may also be incorporated into the subject protocol, to take advantage of the more conventional tolerance strategy of achieving hematopoietic chimerism or microchimerism.

Administration of the lymphocyte depleter and deoxyspergualin compound may begin either before or at the time of the transplant, or in appropriate cases after the transplant. As a practical matter, the timing of administration will be dictated to some extent by the nature of the graft, i.e., whether it is cadaveric or living-related, as advanced planning for cadaveric transplants can be difficult. The immunsuppressant taper will generally begin shortly before or after the transplant and may continue for a fixed period of time as necessary to guard against acute rejection. Alternatively, the immunsuppressant taper can be reduced and eliminated as determined empirically by the transplant clinician based on continual monitoring of appropriate biological markers for graft function and viability.

The appropriate dosages of the subject agents, e.g., the lymphocyte depleter, deoxyspergualin compound and mTOR inhibitor can be easily determined by one of ordinary skill in the art. The dose may vary depending on the age, health and weight of the recipient, the type and nature of the transplant, kind of concurrent treatment, if any, and the frequency of such treatment.

The preferred doses of the lymphocyte depleter are those sufficient to deplete peripheral blood T-cell levels to 80%, preferably 90%, and more preferably 95% or higher of preinjection levels. Generally, when the lymphocyte depleter comprise an ATG, the dosage will range from about 0.01 up to 30 mg/kg total dose during the first 21 days post-transplant, and may include dosing for up to 7 days pre-transplant. Preferably, the dose will be about 10-15 mg/kg total dose over the first 10-14 days post-transplant.

Exemplary doses for the deoxyspergualin compound range from 0.01 up to 3 mg/kg/day for up to 35 days of therapy starting as early as day −7 pre-transplant and going up to 28 days post-transplant. Preferably, the dosage will be between 0.1 and 1 mg/kg/d for days −1 to day +14.

Exemplary doses for the mTOR inhibitor will generally range from about 0.001 to 2 mg/kg body weight, preferably about 0.01 to 1 mg/kg body weight, and more preferably about 0.1 to 0.5 mg/kg body weight, and may be empirically determined by the skilled clinician. Effective plasma concentrations of rapamycin will typically vary from 5 to 20 ng/ml depending on the combination of agents used. Preferably, the mTOR inhibitor is administered for up to 18 months post-transplant, more preferably from 6 to 12 months post-transplant, and ideally from one to six months post transplant, with a gradual reduction or taper of the administered amount over the time course of administration. It should be noted that the dose of any or all of the subject agents given to one subject may vary during the course of the treatment.

In another embodiment, the present invention provides improved methods for treating graft versus host disease. As noted above, GVHD is a morbid complication of bone marrow transplantation, which is often utilized as front-line therapy for various leukemias and lymphomas. Successful donor T cell depletion techniques have been associated with a higher frequency of graft rejection and leukemia relapses (Gale and Butturini (1988) Bone Marrow Transplant 3:185; Devergie et al. (1990) ibid 5:379; Filipovich et al. (1987) Transplantation 44). Therefore, the donor T cells appear to aid engraftment and to provide a graft-versus-leukemia effect as well as causing GVHD. Because the T cell burden following bone marrow transplantation is low for the first 14 days (<10% of normal) the log kill of donor T cells would be proportionally enhanced (Marsh and Neville (1987) Ann. N.Y. Acad. Sci. 507:165; Yan et al., submitted; Gale and Butturini (1988) Bone Marrow Transplant 3:185; Devergie et al. (1990) ibid 5:379; Filipovich et al. (1987) Transplantation 44). It is expected that donor T cells can be eliminated at set times during the early post transplantation period using the present method. In this way the useful attributes of grafted T cells might be maximized and the harmful effects minimized.

The pharmaceutical compositions of the present invention can be included in a container, pack or dispenser together with instructions for administration. Kits for practice of the instant invention are also provided, which may come packed with appropriate amounts of the subject agents together with instructions for use.

### EXPERIMENTAL METHODS

Objective 1: Determination of Whether ATG and LF in Combination Therapy Exhibit a Synergistic Effect on Renal Allograft Survival and Induce Durable Donor Specific Tolerance in Non-Human Primates

Animal model

Kidney transplantation is performed in ABO compatible cynomolgus monkeys. Allograft status is confirmed by MHC typing. The native kidneys of transplant recipients are removed during transplantation in order to produce a life-supporting model.

Transplant Recipient Cynomolgus Monkey Groups (1-4)

Group 1: Untreated control (n=3)

Group 2: LF; dose is 0.1 mg/kg/day; for days 0-14, s.c. (n=4)

Group 3: ATG; dose is 1 mg/kg/day on day 0; 5 mg/kg/day on days 1 and 2; and 10 mg/kg/day on days 3 and 4 (n=4)

Group 4: LF+ATG; LF dose is 0.1 mg/kg/day for days 0-14, s.c.; ATG dose is 1 mg/kg/day for days 0-7, I.V. (n=8).

Assessment of Transplant Recipient Cynomolus Monkey Groups (1-4)

1) Survival.

2) Histology: Biopsies are performed on day 7 and every two months subsequent to day 7.

3) Immunopathology: CD3, CD4, CD8, CD20, IgG, IgM, C3.

4) FACS: CD3, CD4, CD8, CD45, CD20 etc. are performed weekly during the first month and are performed monthly after the first month.

5) Skin Grafting: Animals which survive for 1 year receive skin grafts from the donor as well as from a third party.

Objective 2: Determination of Whether Tolerance Induction by an ATG+LF Combination Therapy Can Be Augmented by Sirolimus (Rap)

The additive effect of Rapamycin on ATG+LF tolerance induction in the primate model is examined.

Protocol

Group 1: RAP monotherapy; RAP is given at a dose of 0.1 mg/kg s.c. for 14 days (n=4).

Group 2: ATG+LF (regimen identical to regimen used in Experiment 1)+RAP (regimen identical to regimen used for Group 1).

The animal, surgical models, and assessment are identical to the animal, surgical models, and assessment described in Objective 1. The following additional assays are performed:

A. RAP Level Measurement: RAP level is measured by HPLC to maintain the trough level of 10-15 ng/ml.

B. Additional groups are added to assess the effect of long term Rap or CyA therapy on graft survival, and the effect of Rap or CyA discontinuation at 3 and 6 months. Initial clinical studies will likely involve the use of low dose maintenance immunosuppression agents.

Detailed Methodolgy

Animals

Outbred juvenile cynomolgus monkeys, sero-negative for simian immunodeficiency virus, and herpes B virus, are purchased from Biomedical Resource Foundation, Houston. The animals are kept in the Primate Facility, The University of Western Ontario. This facility is certified by the Canadian Council on Animal Care (CCAC) for conducting transplant research in non-human primates. Animals are monitored daily. Water and food intake, urine output, as well as frequency and formation of stool are recorded. Animal body weight, temperature, and routine hematology are measured weekly. Signs related to renal failure (tremor, diarrhea, vomiting, dehydration) or to potential side effects from mAb (fever, seizures, rigors, labored breathing) are recorded.

MHC Typing and Donor-Recipient Selection

Donor-recipient combinations are selected based on genetic non-identity at MHC class II. This is established based on denaturing gradient gel electrophoresis and direct sequencing of the second exon of DRB. T-cell responsiveness of the recipient towards the donor is confirmed in vitro for all donor-recipient pairs using the MLR assay. Each monkey is tested against all potential donors to establish the highest responder pairs for transplantation.

Renal Allografts.

The monkeys are heparinized (100 units/kg) during organ collection and implantation. The allograft is implanted using standard microvascular techniques to create an end-to-side anastomosis between the donor renal artery and recipient distal aorta as well as between the donor renal vein and recipient vena cava. A primary ureteroneocystotomy is then created. Bilateral native nephrectomy is completed before closure. Skin sutures are removed after 7-10 days. Monkeys are killed when they develop anuria or have weight loss of 15% of pre-transplantation body weight, in accordance with CCAC standards. Complete gross and histopathological analysis is done at necropsy on all monkeys killed. The experiments described in this study are undertaken according to the principles set forth in the “Guide for the Care and Use of Laboratory Animals”, Canadian Council on Animal Care.

Physiological and Hematological Monitoring.

Serum creatinine, whole blood electrolytes (Na+, K+, Cl−, HCO2−, Ca++), total protein, hemoglobin, hematocrit, leukocyte count and differential, and platelet count are determined at least weekly. Monkeys are weighed weekly. Lymphocyte phenotype is assessed by flow cytometry pre-operatively and on a weekly basis post-operatively, as described. Serological tests for CMV antibodies are done on sera using the IMX System, CMV IgG kit (Abbott Laboratories, Abbott Park, Ill.).

Donor-specific IgG is assessed on whole blood samples pre-operatively and 4 months post-operatively. Donor whole blood is incubated with undiluted recipient sera for 30 min. Third-party and autologous sera and cells are used as negative controls. Cells are then counterstained with antibody against CD3-PE to segregate antibody responsiveness to resting T cells from that of generalized donor-specific antibody. Red cells are lysed with ACK lysis buffer (Biofluids, Rockville, Md.) before analysis on a Becton Dickinson FACScan. Positive samples are tested using serially diluted serum.

MLR

Unidirectional MLRs are done pre-operatively as described. Whole blood is lysed using ACK lysis buffer. The remaining peripheral blood mononuclear cells are washed with PBS then resuspended in RPMI-1640 cell culture media (Life Technologies) supplemented with 10% heat-inactivated fetal calf serum, 2 mM L-glutamine, and penicillin/streptomycin. Gamma-irradiated (50 Gy) donor cells (1×105 per well) serve as stimulators and are co-cultured at a ratio of 1:1 with responder cells. Donor cells for early studies are derived from mesenteric lymph node lymphocytes that have been procured at the time of kidney donation and frozen in freezing media (10% DMSO, 20% autologous serum, 70% RPMI). For later studies evaluating the MLR over time, fresh donor and recipient blood is drawn and the lymphocytes are obtained through Ficoll centrifugation. MLRs are then set up with a constant number of stimulators (1×105 per well), and a varying number of responders (3:1 to 0.1:1). MLRs are assessed every 2 weeks on the later group 1 monkeys to determine the time course to loss of MLR reactivity. The cultures are incubated at 37° C. for 5 days, then pulsed with 10 μCi of H3 thymidine and incubated for an additional 24 h. The MLRs are collected onto a pressed glass filter and counted in a Wallac 1205 beta-plate liquid scintillation counter.

Histology

Renal biopsies are performed on monkeys using ketamine (‘chemical restraint’). Tissues are obtained using a 20-gauge Biopsy-cut needle (C. R. Bard, Covington, Ga.). Tissue samples are embedded in OCT compound (Tissue Tek/Sakura Finetek, Torrance, Calif.), ‘snap-frozen’ in a dry ice/isopentane bath, and stored at −70° C. Frozen sections 6 μm in thickness are stained with hematoxylin and eosin, using standard histological techniques. Immunohistochemistry uses frozen tissue sections 6 μm in thickness adhered to microscope slides (Superfrost Plus; VWR Scientific, West Chester, Pa.), fixed in acetone (HPLC grade; Sigma) for 10 min at −20° C. Slides are then dipped in tap water, then acetone, and allowed to air-dry for at least 1 hour. Endogenous tissue peroxidase is bleached for 1 h at 37° C. using a filtered glucose oxidase solution (0.18 g beta-D(+) glucose (Sigma), 0.005 g glucose oxidase (Sigma), 0.0065 g sodium azide, 50 ml PBS). Slides are washed in PBS for 5-10 min, then loaded onto the Ventana NexES automated immunostainer (Ventana Medical Systems, Tucson, Ariz.). Primary antibody is incubated for 32 min with a chamber temperature of 37° C., avidin/ibotin blocking steps are performed, and the Ventana biotinylated goat anti-rabbit and anti-mouse secondary antibody is added, supplemented with 4% normal horse serum (Vector Labs, Burlingame, Calif.) and 5% normal monkey serum (generated in our facility). Hematoxylin (2 min) and blueing (2 min) steps are completed following the manufacturer's recommendations (Ventana Medical Systems, Tucson, Ariz.). Slides are then water-washed, dehydrated with ethanol, cleared in xylene and coverslipped.

Biopsy RNA Analysis

Total cellular RNA is extracted from ‘snap-frozen’ renal biopsy samples as described. The quality of the RNA is established by visualization in ethidium bromide-stained and agarose gels. Random hexamer-primed (0.1 μg/μl; Stratagene Cloning Systems, La Jolla, Calif.), cDNA synthesis is performed in a volume of 50 μl using 50 U Moloney murine leukemia virus reverse transcriptase (Stratagene, La Jolla, Calif.) in the manufacturer's recommended buffer with 1 mM dNTPs at 37° C. for 60 min. An additional 50 U enzyme is added and transcription continued for an additional 45 min. The enzyme is then inactivated at 65° C. for 10 min before samples are stored at −20° C. Semiquantitative PCR uses primers specific for monkey genes of interest, with the ‘hot start’ method (Molecular Bio-Products, San Diego Calif.), in a volume of 60 μl. The final reaction mix includes standard PCR buffer (Stratagene, La Jolla, Calif.), MgCl (2.5 mM), primers (25 pM/reaction) and dNTPs (0.1 mM). All reactions are amplified on a Perkin Elmer 4800 thermal cycler for 33 cycles, except actin, which is amplified for 28 cycles (94° C. for 1 min; 63° C. for 30 s, and 72° C. for 30 s). Primers are selected for homogenous annealing characteristics.

Enzyme-Linked Immunosorbent Assay (ELISA)

The design of the donor-specific solid phase ELISA assay for testing for the presence of anti-MHC Class I and Class II allo-antibodies in sera is modeled after a commercial kit (SangStat, Menlo Park, Calif.) that is no longer on the market. Donor antigen is prepared from either fresh or frozen splenocytes. Briefly, isolated donor lymphocytes suspended in PBS are incubated with a solubilizing detergent specific for proteins in which the cell membrane is destroyed. Donor MHC molecules are then semi-purified with a saturated 95% ammonium sulfate precipitation. The solution is centrifuged 2× and the precipitate is discarded. The supernatant, containing both solubilized donor MHC Class I and II antigen, is diluted in 1% BSA/PBS. Class specificity is obtained by using the donor soluble antigen lysate on ELISA plates previously coated with either a pan MHC monoclonal antibody directed towards a MHC Class II molecule domain. The plates are incubated for 1 hour at RT and subsequently washed 3× with wash buffer. Recipient sera, pre-treated with a 1:50 dilution of mouse serum and incubated at RT for 60 minutes, is diluted 1:100 with 0.5% BSA/wash buffer and 100 ul is added to appropriate wells. The plate is incubated for 1 hour at RT and washed 3× with buffer. Horseradish peroxidase conjugated with goat anti-human/mouse IgG is added to each well and incubated 30 minutes at RT. The plate is washed 3× with buffer and bound antibody is detected using 0-phenylenediamine dihydrochloide. The reaction is stopped with the addition of 1 N HCL and the absorbance is read on an ELISA plate reader at a 495/630 wavelength. The presence and amount of specifically bound mouse/human IgG is determined by measuring the absorbance in wells containing solubilized donor antigen and subtracting the absorbancy of the non-specific binding in wells without solubilized donor antigen. The value is compared to the positive/negative cut-off value, which is obtained by averaging the individual results obtained from non-sensitized males ≅3 standard deviation. To detect cytokines produced by CD4+ T cells, the supernatants of MLR are harvested after 24-48 hrs culture. The coating antibodies against human IL-2, INF-g, IL-4 and IL-10 (PharMingen) are diluted at 1 mg/ml in coating buffer and are duplicately added 100 ml to each well. After over night incubation, the supernatants are added (100 ml/well) and incubated at room temperature for 1 h. The detecting antibodies are biotin-labeled mAb against same cytokines (ParMingen). After 1 h incubation at room temperature, HRP-conjugated streptavadin (1:5000, 100 ml/well) is added. The reaction is visualized by adding TMB substrate and detecting the absorbency at 450-550 nm.

Enzyme-Linked Immunospot Assay (ELISPOT)

ELISPOT is for detection of B cells that produce donor-reactive allo-antibodies. Murine and human lymphoid cells (from lymph nodes, spleen or peripheral blood) are cultured with or without specific antigens (subcellular alloantigen, TT, or EBV) for 24 hours (to boost IgG production by differentiated B cells) or 7-10 days (to stimulate undifferentiated B cells to secrete IgG). The cultured cells are placed at various dilutions in ELISPOT microtiter plates previously coated with appropriate antigen (TT, EBC). For detection of allo-antibodies, the plates are coated first with anti-mouse or anti-human MHC class I or MHC class II antibodies and then with the subcellular donor alloantigen (similar to the ELISA methodology). After 24 hours, the cells are washed from the plates, and the plates are developed with an enzyme-linked goat anti-human or mouse IgG antibody plus appropriate substrate. The Elispot plates are evaluated with a Zeis Elispot reader. Initially, optimal conditions are determined under which individuals who are known seropositive for any of the test antigens, have corresponding positive, antigen-specific ELISPOT results.

Real-Time PCR for mRNA Quantification

This technique uses flanking nucleotide primers and an oligonucleotide hybridization probe that is labeled with a reporter fluorescent dye (6-carbosy-fluorescein) at the 5-end and with a quencher fluorescent dye (6-carbosy-tetramethylrhodamine) at the 3-end. The sense and antisense of each pair of primers are designed for different exons of the gene, to eliminate detection of genomic DNA. Before starting the PCR reaction, the probe is intact and the reporter dye emission is quenched due to its physical proximity to quencher fluorescent emissions. During the extension of the phase of the PCR cycle, however, the nucleolytic activity of the Taq DNA polymerase cleaves the hybridization probe and releases the reporter dye, thus producing detectable reporter dye emission. The resulting relative increase in reporter fluorescent dye emission is monitored in real-time PCR amplification using the Sequence Detection System (ABI PRISM 7700 Sequence Detection System and software, PE Applied Biosystems, Inc., Foster City, Calif.). PCR is performed in a 25 ml reaction volume that contains 2.5 ul of cDNA from the RT reaction, primers/probes mixture (900 nM of each sense and antisense primers and 120 nM of FAM-labeled probe), and 12.5 ml of the 2 MM buffer from Perkin Elmer (Norwalk, Conn.). The 18S ribosomal RNA is used as an internal standard to control for variability in amplification due to differences in starting mRNA concentrations. Sense and antisense primers (0.5 nM) and JOE-labels probe for 18S rRNA are added to each reaction. The level of the cytokine MRNA, relative to 18S rRNA, is calculated using the formula: Relative mRNA expression =2−(Ct of cytokine-Ct of 18S)×1010. Where Ct is on the threshold cycle value. Since the levels of cytokine mRNA are considerably lower than the level of 18S rRNA, all mRNA values shown are arbitrarily multiplied by 1010. Cytokine mRNA levels are reported as fold-changes over background levels detected in control tissues. These changes in cytokine mRNA expression levels are graded on the following scale: (−)=background or lower levels; (+)=2 to 100 fold increase over background; (++)=100 to 1000 fold increase over background; (+++)=>1000 fold increase over background

Flow Cytometry

Flow cytometry of peripheral blood are used to: 1. Show the binding of antibody to cells; 2. Measure the excess of antibody; 3. Show shifts in the expression of CD45RB; 4. Record the changes of cell populations; and 5. Measure the presence of monkey-anti-mouse antibodies. Heparinized blood and serum are collected according to protocol. Mononuclear cells are isolated by Ficoll-Paque density centrifugation and 0.2-0.5×10⁶ cells are used per test. As wash and incubation buffer PBS containing 1% FBS and 0.1% sodium azide is used and for fixation of cells 2% paraformaldehyde in PBS. Incubations with antibodies take place at 4° C. for 30 minutes followed by a wash. Samples are analyzed on a flow cytometer (FACScan, BD sciences). For (1) the antibody used is anti-human IgG2 PE conjugated (Southern Biotech). For (2) monkey serum is used on normal monkey lymphocytes with anti-human IgG2-PE as a secondary antibody. For (3) the chimeric 6G3 antibody is used with anti-human IgG2-PE as the secondary antibody. For (4) several antibodies are used: anti-CD3 to show total T cells; CD20 for total B cells; CD14 to measure monocyte counts. The different T cell subsets, activation stage, and costimulatory markers are analyzed with CD4 and CD8, CD25 and CD69, CD28 and CTLA4. All antibodies are commercially available with fluorescent labels (Pharmingen, BD Sciences). Test (5) uses serum to bind to mouse splenic lymphocytes with goat-anti-monkey IgG-FITC (Serotec) as secondary antibody. All comparisons are done within the same monkey before transplant samples and sequential samples after transplant.

DC Purification

To isolate CD11C+ DC, peripheral blood mononuclear cells (PBMC) from monkeys are isolated by a Histopaque density-gradient centrifugation. The PBMC are positively isolated by incubation CD11C mAb labeled MACS beads. Ten million PBMC (in 90 ml of PBS supplemented with 0.5% bovine serum albumin) are incubated with 10 ml of anti-human CD11C conjugated beads at 6° C. for 15 min. CD11C+ DC are isolated and purified by a positive selection column. DC from tolerant monkey also can be augmented by in vitro culture. CD11C+ DC from above isolation, CD34+ progenitors from PBMC or bone marrow-derived precursor cells are cultured in 24-well plates (2×106 cells per well) in 2 ml RPMI 1640 supplemented with 10% fetal-calf serum (FCS, Gibco RBL), 100 U/ml of penicillin, 100 mg of streptomycin, 2-mercaptoethanol (50 mM, Gibco RBL), recombinant human GM-CSF (10 ng/ml; Peprotech, Rocky Hill, N.J.) and recombinant human IL-4 (10 ng/ml; Peprotech). All media and additives are documented to be free of LPS contamination. Non-adherant granulocytes are removed after 48 hrs of culture and fresh media added every 48 hrs. By day 4-6 of culture, proliferating clusters of cells with typical dendritic morphology are seen. By day 7-9 in culture >90% of the cells express the DC specific marker DEC-205. The proportion of cells staining for T (CD3) and B (B220) lymphocytes are consistently <3% based on observation. The phenotype of surface molecules in mature DC are CD11C+, DECD205+, MHC II high, CD40+, CD86+. To obtain fully mature DC, 10 ng/ml LPS plus 10 ng/ml TNF-a, or 1 mg/ml anti-CD40 Ab are added on day 7. These cells are used in in-vitro assays.

DNA Microarray

Tissue samples from biopsies are snap frozen and RNA extracted using RNeasy Midi columns (Qiagen Inc., Mississauga, ON). In general, 200-300 mg of total RNA can be isolated from a 0.5 cm³ tissue section and 50-70 mg of total RNA can be isolated from a needle biopsy. Current protocols can use as little as 1 ug of RNA. The micro-array protocols are adapted from the standard methods of the Clinical Genomics Center at the Ontario Cancer Institute in Toronto, Ontario (http://www.uhnres.utoronto.ca/services/microarray). Briefly, fluorescently labeled cDNA probes are made starting with 1-10 mg total RNA using Superscript II reverse transcriptase (Life Technologies, Burlington, ON) and FluoroLink Cy3- and Cy5-dCTP dyes (Amersham Pharmacia Biotech, Buckinghamshire, England). Both combinations of dye incorporation are used with respect to each sample pair to compensate for unequal dye incorporation. Known concentrations of Arabidopsis thaliana RNA are used in the reverse transcription reaction for internal quantitation standards. RNA sample/probes will be combined with control RNA samples/probes and added to hybridization solution (DIG Easy Hyb, Roche, Laval, QC) before being applied to the microarrays for an overnight incubation at 37° C. in a humidified slide box chamber. The high degree of DNA identity (>94%) between human and cynologous monkey genes (coding and 3′ UTRs) allow for efficient hybridization of nonhuman primate probes to human DNA microarrays. Following washes in SSC/0.1% SDS (3×10 minutes at 50° C.) and SSC (2×10 seconds at 50° C., the microarrays are dried in a centrifuge then scanned on a Virtek ChipReader (Virtek Inc., Waterloo, ON). Experimental RNA sample/probes (Cy3 or Cy5) from each experimental group are combined with a control RNA sample/probe (Cy3 or Cy5) derived from intact kidneys for hybrizidization to DNA microarrays. The native kidney samples serves as a baseline standard so that all experimental groups can be compared to each other. Experimental/control samples are hybridized with two DNA microarrays. Previous studies used the 19.2K microarray containing 19200 human genes from the Clinical Genomics Center, Ontario Cancer Institute, Toronto, ON. The 19.2K DNA micro-array contains approximately 1,300 of the 2,400 known immune related and injury related genes. It also contains several genes of unknown function. Thus the 19.2K DNA microarray is well suited for screening of both immune and injury related genes and determining if genes with unknown function are involved in tolerance or rejection. To complement the 19.2K DNA microarray the newly designed immune/injury related DNA micro-array developed through a collaborative effort between the TransNet consortium, the CanVac consortium and the Clinical Genomics Center is used. The immune/injury related gene DNA micro-array contains all known immune related/injury related genes (2,600). Data analysis is performed on the raw data sets using QuantArray analysis software (GSI Lumonics, Billerica, Mass.), custom statistical and stringency software, and Spotfire Pro 4.0 visualization software (Somerville, Mass.). Data is further compiled using cluster analysis software and available through the TransNet website.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method of prolonging the survival of a transplant allograft in a recipient, said method comprising administering to said recipient simultaneously or sequentially a therapeutically effective combination of a lymphocyte depleter, a deoxyspergualin compound and an mTOR inhibitor.

2. The method according to claim 1, wherein said lymphocyte depleter is selected from the group consisting of an anti-thymocyte globulin, an anti-lymphocyte globulin, a T cell immunotoxin, and a monoclonal antibody preparation directed against a lymphocyte surface protein.

3. The method according to claim 2, wherein said lymphocyte depleter comprises an anti-thymocyte globulin.

4. The method according to claim 3, wherein said anti-thymocyte globulin comprises a rabbit polyclonal anti-thymocyte globulin.

5. The method according to claim 2, wherein said lymphocyte depleter comprises a monoclonal antibody preparation directed against a lymphocyte surface protein, and wherein said lymphocyte surface protein comprises CD52.

6. The method according to claim 1, wherein said deoxyspergualin compound is selected from the group consisting of 15-deoxyspergualin, LF15-0195, LF-08-0299, and methyldeoxyspergualin.

7. The method according to claim 1, wherein said mTOR inhibitor comprises a rapamycin compound.

8. A method for inducing immune tolerance to an antigen in a recipient host, comprising the simultaneous or sequential administration of a lymphocyte depleter and a deoxyspergualin compound to the host, in combination with the administration of an immunosuppressant taper.

9. The method according to claim 8, wherein said immunosuppressant taper comprises the tapered administration of an mTOR inhibitor to said host.

10. The method according to claim 9, wherein said mTOR inhibitor comprises a rapamycin compound.

11. The method according to claim 8, wherein said lymphocyte depleter is selected from the group consisting of an anti-thymocyte globulin, an anti-lymphocyte globulin, a T cell immunotoxin, and an monoclonal antibody preparation directed against a lymphocyte surface protein.

12. The method according to claim 11, wherein said lymphocyte depleter comprises an anti-thymocyte globulin.

13. The method according to claim 12, wherein said anti-thymocyte globulin comprises a rabbit polyclonal anti-thymocyte globulin.

14. The method according to claim 8, wherein said deoxyspergualin compound is selected from the group consisting of 15-deoxyspergualin, LF15-0195, LF-08-0299, and methyldeoxyspergualin.

15. The method according to claim 8, wherein said antigen is a host antigen.

16. The method according to claim 8, wherein said antigen is a donor antigen.

17. The method according to claim 16, further comprising the administration to said host of donor hematopoietic cells to induce hematopoietic chimerism or microchimerism.

18. A method for treating GVHD in a recipient host, comprising the simultaneous or sequential administration of a lymphocyte depleter and a deoxyspergualin compound to the host, in combination with the tapered administration of an mTOR inhibitor.

19. A kit for use in the treatment of a transplant recipient, said kit comprising a lymphocyte depleter, a deoxyspergualin compound and an mTOR inhibitor.
